SEQUENCE LISTING (8 sequences)

SEQ ID NO: 1 - RNA, 19 nt, Artificial Sequence
  sense strand of anti-luciferase siRNA
ccacacuauuuagcuucuu

SEQ ID NO: 2 - RNA, 19 nt, Artificial Sequence
  antisense strand of anti-luciferase siRNA
aagaagcuaaauagugugg

SEQ ID NO: 3 - RNA, 21 nt, Artificial Sequence
  sense strand of siNRA against Apolipoprotein B
gucaucacacugaauaccaau

SEQ ID NO: 4 - RNA, 21 nt, Artificial Sequence
  antisense strand of siNRA against Apolipoprotein B
auugguauucagugugaugac

SEQ ID NO: 5 - DNA, 21 bp, Artificial Sequence
  sense primer for ApoB
ttttcctcccagatttcaagg

SEQ ID NO: 6 - DNA, 22 bp, Artificial Sequence
  antisense primer for ApoB
tccagcattggtattcagtgtg

SEQ ID NO: 7 - DNA, 20 bp, Artificial Sequence
  sense primer for GAPDH
ccttcattgacctcaactac

SEQ ID NO: 8 - DNA, 20 bp, Artificial Sequence
  antisense primer for GAPDH
ggaaggccatgccagtgagc

(19) United States
(12) Patent Application Publication
(10) Pub. No.: US 2014/0080897 A1
(43) Pub. Date: Mar. 20, 2014

(54) HYALURONIC ACID-NUCLEIC ACID CONJUGATE AND COMPOSITION FOR NUCLEIC ACID DELIVERY CONTAINING THE SAME

(71) Applicant: POSTECH ACADEMY-INDUSTRY FOUNDATION, Pohang-si (KR)

(72) Inventors: Sei Kwang HAHN, Pohang-si (KR); Kitae Park, Jeonju-si (KR); Jeonga Yang, Pohang-si (KR); Won Ho Kong, Pohang-si (KR); Min Young Lee, Pohang-si (KR)

(73) Assignee: POSTECH ACADEMY-INDUSTRY FOUNDATION, Pohang-si (KR)

(21) Appl. No.: 14/027,391

(22) Filed: Sep. 16, 2013

(30) Foreign Application Priority Data
Sep. 18, 2012 (KR) 10-2012-0103521

(51) Int. Cl. A61K 47/48; A61K 31/7088

(52) U.S. Cl. CPC A61K 47/4823; A61K 31/7088
USPC 514/44 A; 536/55.1; 536/24.5; 525/54.2

(57) ABSTRACT

The present invention relates to a hyaluronic acid-nucleic acid conjugate for the development of in vivo nucleic acid delivery system, and the development of nucleic acid delivery system using the same. Specifically, a hyaluronic acid-nucleic acid complex wherein a hyaluronic acid-alkylenediamine conjugate and nucleic acid are connected by a disulfide bond; a composition for nucleic acid delivery comprising the hyaluronic acid-nucleic acid complex as an active ingredient; a method for preparing the hyaluronic acid-nucleic acid complex; and a method for in vivo delivery of nucleic acid, comprising administering the hyaluronic acid-nucleic acid complex to a subject are provided.

### CROSS REFERENCE TO RELATED APPLICATION

The present application claims priority to and the benefit of Korean patent application No. 10-2012-0103521 filed in the Korea Intellectual Property Office on Sep. 18, 2012, the entire content of which is incorporated hereinto by reference.

### BACKGROUND OF THE INVENTION

1. Field of the Invention

The present invention relates to a hyaluronic acid-nucleic acid complex for the development of in vivo nucleic acid delivery system, and the development of nucleic acid delivery system using the same. Specifically, the present invention relates to a hyaluronic acid-nucleic acid complex wherein a hyaluronic acid-alkylenediamine conjugate and nucleic acid are connected by a disulfide bond; a composition for nucleic acid delivery comprising the hyaluronic acid-nucleic acid complex as an active ingredient; a method for preparing the hyaluronic acid-nucleic acid complex; and a method for in vivo delivery of nucleic acid, comprising administering the hyaluronic acid-nucleic acid complex to a subject.

According to a method for preparing a polymer for nucleic acid delivery and a composition for nucleic acid delivery comprising the conjugate of the present invention, nucleic acid may be effectively delivered in the cells compared to the existing technologies, toxicity may be effectively decreased because cationic polymer with relatively low toxicity may be used, and hyaluronic acid with excellent biocompatibility such as liver targetability and skin permeability may be introduced and applied for various nucleic acid delivery methods.

2. Description of the Related Art

Since RNA interference has been discovered by Fire and Mello in 1998, there have been continued attempts to utilize siRNA for the treatment of human diseases. Based on the fact that synthesizable short RNA may inhibit the expression of specific genes, it draws a lot of attentions as a novel therapeutic agent. However, although siRNA is a potent candidate for drugs, its delivery method is a large obstacle to develop as a therapeutic agent. Since negatively charged RNA molecule is difficult to pass through negatively charged cell membrane, intracellular delivery capacity is lowered, and a RNA molecule that is introduced in the body is rapidly decomposed by decomposition enzyme. To overcome these problems, studies have been actively progressed on the development of delivery system using virus or the development of nucleic acid delivery system using a conjugate using cationic lipid carrier or cationic polymer, and the like. Although the delivery system using virus exhibit very high delivery efficiency, it has a disadvantage in that application to human is very limited due to stability problem of virus.

Accordingly, as a safer alternative, the development of delivery system using cationic polymer or lipid body draws attentions. It has been reported that cationic material forms a conjugate (polyplex) with negatively charged nucleic acid material through charge complementarity to protect siRNA from decomposition enzyme, facilitate intracellular delivery, and deliver siRNA to cytoplasm based on pH buffering action in the intracellular endosome. However, cationic polymer is known to have disadvantages in that it induces necrosis and apoptosis, and if delivered in the body, it causes cell aggregation and the delivery efficiency is lowered due to bonding with blood protein. To overcome these, the development of delivery system using cationic polymer having low molecular weight or linear polymer is being considered, but it may not be easily bonded to siRNA due to low charge, and it has low delivery efficiency, thus having a limit to be developed as delivery system.

### SUMMARY OF THE INVENTION

It is an object of the present invention to provide a more efficient nucleic acid delivery system by facilitating the formation of a conjugate of nucleic acid and cationic material, and weakening the toxicity of the conjugate.

Specifically, one embodiment provides a conjugate of hyaluronic acid (HA) and alkylenediamine.

Another embodiment provides a hyaluronic acid-alkylenediamine-nucleic acid complex wherein the hyaluronic acid (HA)-alkylenediamine conjugate and nucleic acid are connected by a disulfide bond.

Still another embodiment provides a composition for nucleic acid delivery comprising the hyaluronic acid-alkylenediamine-nucleic acid complex as an active ingredient.

Still another embodiment provides a method f preparing the hyaluronic acid-alkylenediamine-nucleic acid complex.

Still another embodiment provides a method for in vivo delivery of nucleic acid, comprising administering the hyaluronic acid-alkylenediamine-nucleic acid complex to a subject.

### BRIEF DESCRIPTION OF THE DRAWINGS

FIG. 1 shows a synthesis method by reacting hyaluronic acid and alkylenediamine to introduce an amine group in the hyaluronic acid.

FIG. 2 shows the analysis result of the synthesis of HA-DAB using 1H NMR.

FIG. 3 shows a synthesis method of a HA-nucleic acid conjugate comprising a disulfide bond using HA-DAB and SPDP.

FIGS. 4a and 4b show the analysis results of the synthesis of HA-DAB-siRNA (4a) or HA-siRNA (4b) using HPLC.

FIGS. 5a to 5d show the results of confirming the separation of HA-DAB-siRNA by a reducing agent through agarose gel electrophoresis.

FIG. 6 shows the result of confirming the toxicity of HA-DAB-siRNA using MDA-MB-231 cell line.

FIGS. 7a and 7b show the measurement results of luciferase expression by treating luciferase-expressing MDA-MB-231 cells with various ratios of HA-DAB-siRNA/LPEI complexes.

FIGS. 8a and 8b respectively show the particle size (8a) and the surface charge (8b) of HA-DAB-siRNA/IPEI particles.

FIG. 9 shows the measurement result of luciferase activity after treating MDA-MB-231 cells with a cellular uptake inhibitor and a HA-DAB-siRNA conjugate.

FIG. 10 shows the target gene inhibition activity of a HA-DAB-siNRA conjugate in liver tissue.

FIG. 11 shows the result of confirming the formation of a HA-DAB-siRNA/CSLN complex through agarose gel electrophoresis.

FIGS. 12a and 12b show the results of confirming the toxicity of a HA-DAB-siRNA/CSLN complex by MTT using Hela cell line.

FIGS. 13a and 13b show the measurement result of the concentration of mFVII secreted in the supernatant, after treating a HA-DAB-siRNA/CSLN complex.

### DETAILED DESCRIPTION OF THE PREFERRED EMBODIMENTS

The present invention is to solve high toxicity and low delivery efficiency of cationic material, which have been pointed out as problems of the existing nucleic acid delivery system using polymer. Specifically, it has been reported that polyethyleneimine (PEI) widely known as a nucleic acid delivery vector, if it is a branched polymer, exhibit high delivery efficiency but has high toxicity in cellular and in vivo experiments due to strong cationic property. Alternatively, a linear type or a low molecular type has been suggested, but although they have low toxicity, they cannot easily form a conjugate with nucleic acid material and have very low delivery efficiency.

Thus, the inventors, as the result of assiduous studies, introduced anionic polymer hyaluronic acid (HA) with excellent biocompatibility in nucleic acid material, thereby increasing anionic property to facilitate the formation of a conjugate, and neutralizing high positive charge to decrease cellular and in vivo toxicity, and completed the invention.

One embodiment of the invention relates to a hyaluronic acid-alkylenediamine conjugate. More specifically, the hyaluronic acid-alkylenediamine conjugate comprises hyaluronic acid and alkylenediamine, wherein one of the two amine groups of the alkylenediamine may be connected to the carboxylic acid group of the hyaluronic acid. The alkylenediamine and the hyaluronic acid may be connected by a peptide bond. The peptide bond may be formed between the amine group of the alkylenediamine and the carboxylic group of the hyaluronic acid. Since the hyaluronic acid-alkylenediamine conjugate is connected with nucleic acid through a disulfide bond and may stably deliver nucleic acid in the body, it may be useful for nucleic acid delivery. And, by controlling the reaction time of the hyaluronic acid and the alkylenediamine, the substitution rate (i.e. the ratio of the carboxylic acid groups that have reacted with alkylenediamine in the carboxylic acid groups of the hyaluronic acid) may be easily controlled (see Example 1-1). Specifically, since the substitution rate is in proportion to the reaction time of the hyaluronic acid and alkylenediamine, the reaction time may be lengthened if high substitution rate is to be achieved, and the reaction time may be shortened if low substitution rate is to be achieved. Since a binding force is determined by the substitution rate when hyaluronic acid binds to a receptor in the body, the substitution rate of hyaluronic acid may be important in biomaterial delivery. For example, as the substitution rate of hyaluronic acid is higher, hyaluronic acid circulates in the body for a long time without being bound to a receptor, while as the substitution rate of hyaluronic acid is lower, a tendency of hyaluronic acid to bind to a receptor becomes stronger, and thus, targeted delivery to tissue expressing a receptor a lot may be enabled. Thus, desired property may be achieved by appropriately controlling the substitution rate of hyaluronic acid.

Another embodiment relates to a hyaluronic acid-alkylenediamine-nucleic acid complex wherein a hyaluronic acid-alkylenediamine conjugate and nucleic acid are connected by a disulfide bond. In the hyaluronic acid-alkylenediamine-nucleic acid complex, C1-10 alkylenediamine and hyaluronic acid may be connected by a peptide bond, and nucleic acid containing a thiol group may be bound to the amine group of the alkylenediamine directly or through a linker. More specifically, the hyaluronic acid-alkylenediamine-nucleic acid complex may comprise hyaluronic acid, alkylenediamine and thiol group-introduced nucleic acid, wherein one of the two amine groups of the alkylenediamine is connected to the carboxylic acid group of the hyaluronic acid, and the other amine group is connected to the thiol group introduced in the nucleic acid through a disulfide bond. The hyaluronic acid-alkylenediamine-nucleic acid complex may be useful as a composition for in vivo delivery of nucleic acid.

Hereinafter, the present invention will be explained in detail.

The hyaluronic acid (HA), which is anionic mucopolysaccharide, is living body-derived material existing in animal-derived vitreous body, joint fluid, cartilage, skin, and the like, and is a polymer consisting of the unit of the following Chemical Formula 1.

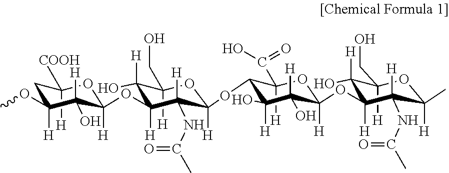

Although the molecular weight of the hyaluronic acid that can be used in the present invention is not specifically limited, the weight average molecular weight may be preferably about 10,000 to 3,000,000.

Although the alkylenediamine used in the present invention is not specifically limited, C1-10, particularly C4-8 alkylenediamine (Chemical Formula 2) is preferable, and for example, it may be selected from the group consisting of 1,4-diaminobutane (DAB), hexamethylenediamine (HMDA), and the like.

H₂N(CH)ₘNH₂ [Chemical Formula 2]

wherein, m is an integer of 1 to 10, preferably an integer of 4 to 8.

One of the two amine groups of the alkylenediamine is connected to the carboxylic acid group of the hyaluronic acid to form a conjugate of the structure of the following Chemical Formula 3.

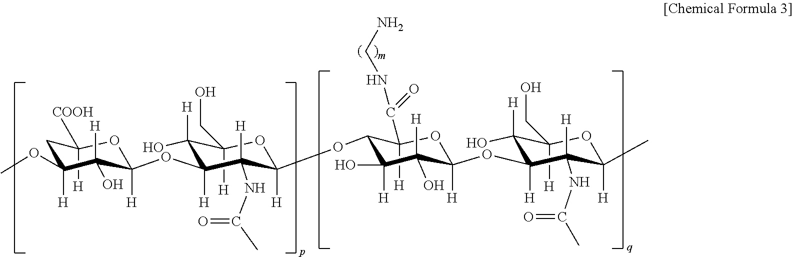

wherein, m is an integer of 1 to 10, specifically an integer of 4 to 8, and p and q are independently an integer selected from 16 to 2500.

A hyaluronic acid-alkylenediamine-nucleic acid complex wherein nucleic acid is bonded to the hyaluronic acid-alkylenediamine conjugate is provided. The nucleic acid is connected to the amine group of the hyaluronic acid-alkylenediamine conjugate through a disulfide bond. Since the disulfide bond may be easily decomposed when delivered into the cytoplasm, connection of the hyaluronic acid-alkylenediamine-nucleic acid complex through a disulfide bond may be favorable for release of nucleic acid (for example, siRNA).

In the hyaluronic acid-alkylenediamine-nucleic acid complex, C1-10 alkylenediamine and hyaluronic acid may be connected by a peptide bond, and nucleic acid containing a thiol group may be bonded to the amine group of the alkylenediamine directly or through a linker. The linker may be derived from a linker compound comprising a first functional group that can be bonded to the amine group of the alkylenediamine, and a second functional group that can be bonded to the thiol group of the nucleic acid. The first functional group may be a carboxylic acid group, and the second functional group may be a thiol group. For example, the linker may be OH—C(O)—(CH₂)s-SH (wherein s is an integer of 1 to 5). The linker compound may include all the materials that can be peptide bonded and disulfide bonded, and for example, it may be succinimidyl 3-(2-pyridyldithio)propionate (SPDP).

Specifically, since one of the two amine groups of alkylenediamine is bonded to the carboxylic acid group of hyaluronic acid and the other is bonded to nucleic acid through a disulfide bond, the hyaluronic acid and nucleic acid forms a complex through alkylenediamine.

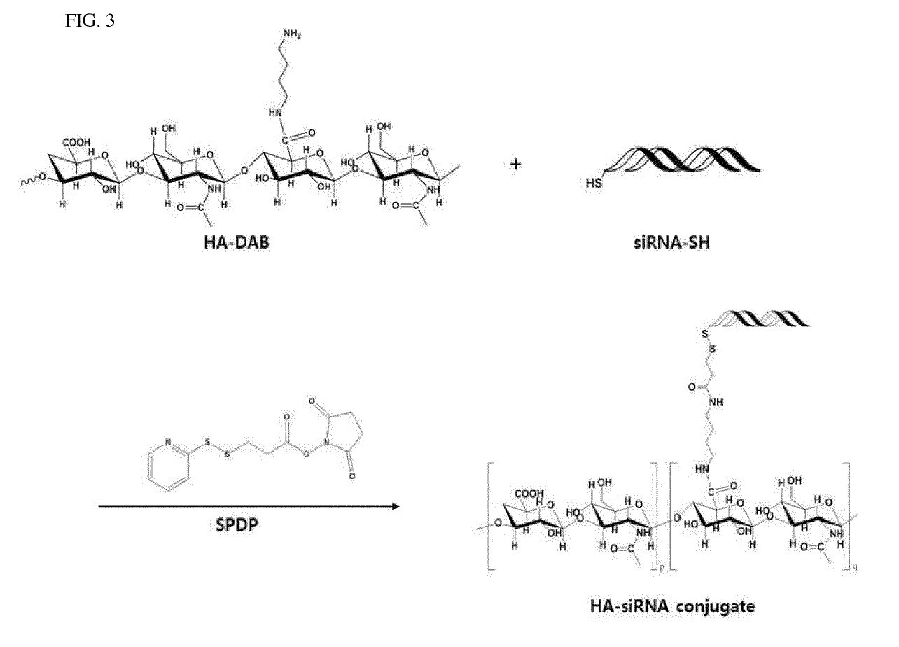

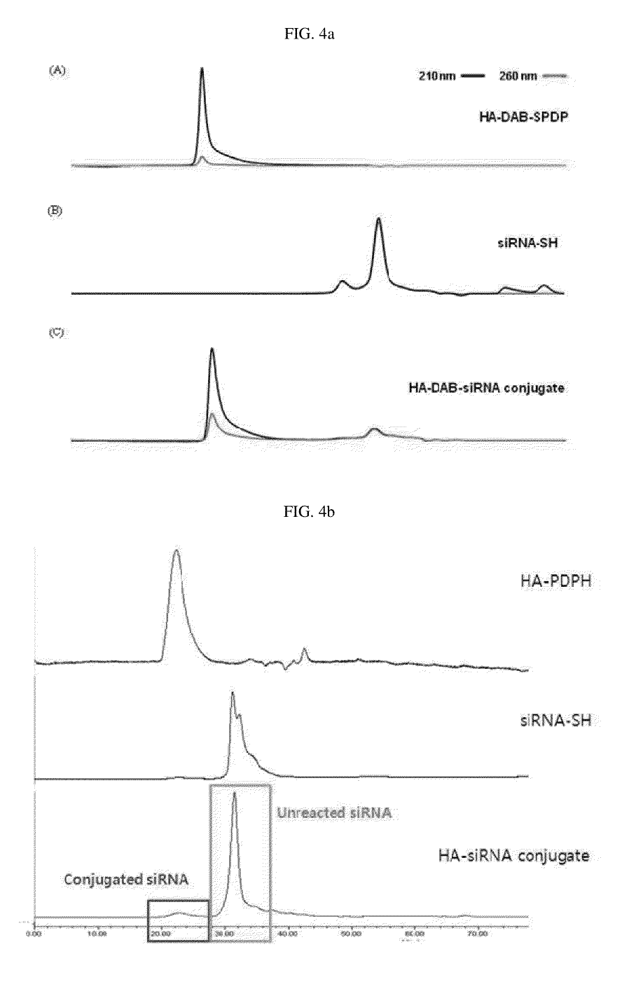

As described above, since hyaluronic acid forms a conjugate through alkylenediamine, production rate of a complex may become remarkably high compared to the case without using alkylenediamine. For example, the production rate of a complex may be about 60% or more, for example about 60-70% in case hyaluronic acid forms a complex through alkylenediamine, while the production rate of a conjugate of hyaluronic acid and nucleic acid is about 20% or less, for example about 10-20% in case alkylenediamine is not used (see Experimental Example 1 and FIGS. 4a and 4b).

The disulfide bond may be formed between nucleic acid and alkylenediamine by the reaction of thiol group-introduced nucleic acid and the hyaluronic acid-alkylenediamine conjugate in the presence of a compound having a thiol group (a linker compound), for example, succinimidyl 3-(2-pyridyldithio)propionate (SPDP), and the like. The thiol group may be introduced in the nucleic acid at a part excluding 5′-OH or phosphate terminus of antisense strand which inhibits siRNA efficiency, specifically it may be preferably introduced at the 3′ end of sense strand, but is not limited thereto.

The nucleic acid may include all kinds of single stranded or double stranded nucleic acids such as deoxyribonucleic acid (DNA), ribonucleic acid (RNA), or polynucleotide derivatives with chemically modified backbone, sugar or base or modified terminal, and the like, and specifically, it may be at least one selected from the group consisting of RNA, DNA, siRNA (short interfering RNA), aptamer, antisense oligodeoxynucleotide (antisense ODN), antisense RNA, ribozyme, DNAzyme, and the like. In the present invention, to introduce a disulfide bond, the nucleic acid may have a thiol group introduced at 3′ end. For example, the nucleic acid may be nucleic acid having a thiol group introduced at the 3′ end of sense strand, for example, siRNA.

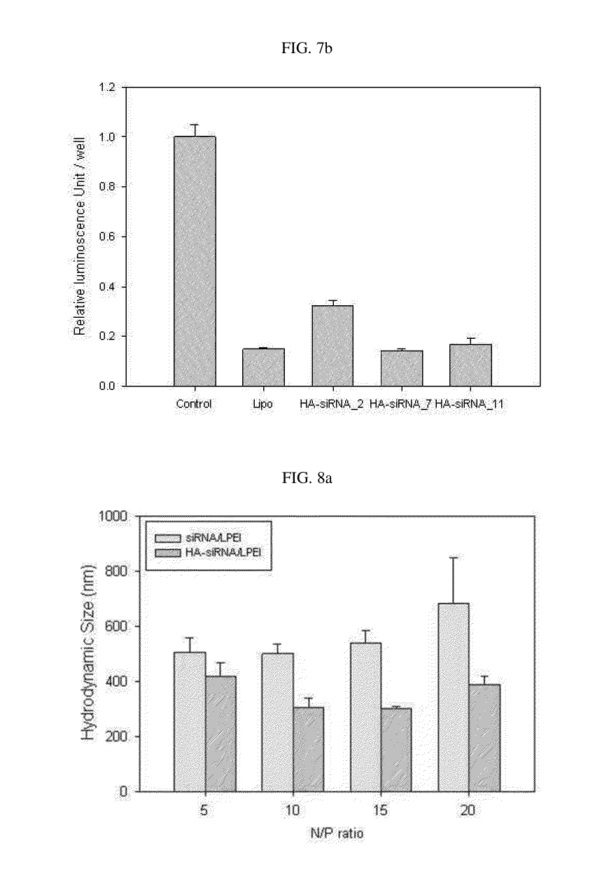

In the hyaluronic acid-alkylenediamine-nucleic acid complex, the number of siRNA conjugated per one hyaluronic acid molecule (for example, molecular weight 100 kDa) may be 2 to 15, specifically 2 to 11 (see FIG. 7b).

The introduction of a thiol group in the nucleic acid may be conducted by methods commonly known in the art.

According to one embodiment of the invention, the hyaluronic acid-alkylenediamine-nucleic acid complex may have a structure of the following Chemical Formula 4 (hyaluronic acid-double stranded nucleic acid (for example, siRNA) conjugate).

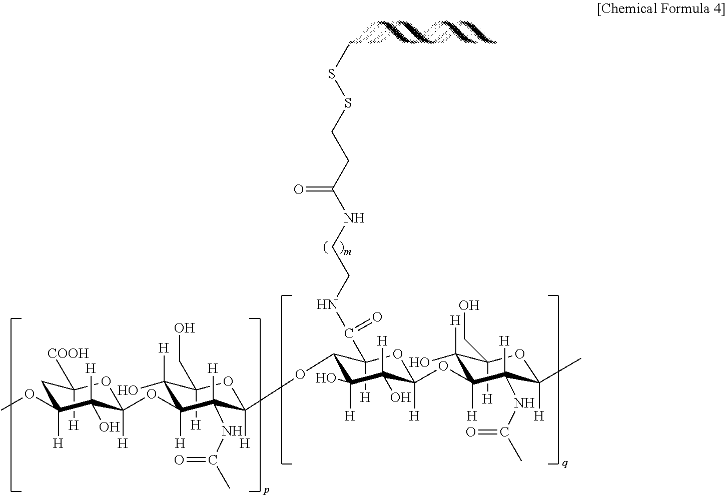

wherein m is an integer of 1 to 10, specifically an integer of 4 to 8, and p and q are independently an integer of 16 to 2500.

For example, if the alkylenediamine is diaminobutane, the hyaluronic acid-alkylenediamine-nucleic acid complex may have a structure of the following Chemical Formula 5:

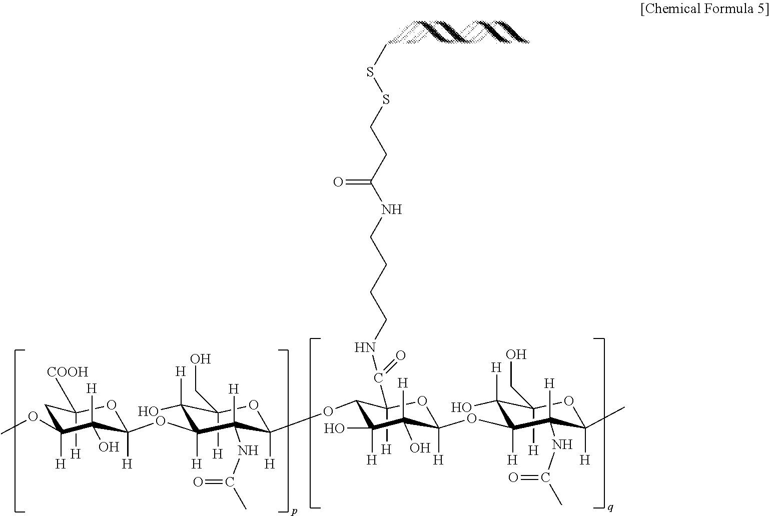

Particularly, since there are a lot of hyaluronic acid receptors in liver tissue, it may be particularly favorable for liver tissue specific delivery.

The hyaluronic acid-alkylenediamine-nucleic acid complex may further comprise cationic material that can contribute to bonding with nucleic acid by electrostatic bonding. The cationic material may be at least one selected from the group consisting of polyethyleneimine (PEI) (for example, linear polyethyleneimine, branched polyethyleneimine), poly(L-lysine), polymethacrylate, chitosan, poly cationic dendrimers (for example, polyamidoamine dendrimers, poly(propyleneimine) dendrimers, poly(L-lysine) dendrimers, and the like), cationic peptides (for example, TAT peptide, Antennapedia Homeodomain Peptide, MPG peptide, Transportan Peptide, protamine, and the like), quantum dot, gold nanoparticles, silica nanoparticles, carbon derivative nanoparticles (for example, carbon nanotube, grapheme, carbon dot, and the like), solid lipid nanoparticles, and the like. For example, the cationic material may be liner polyethyleneimine (linear PEI). The cationic polymer (for example, linear polyethyleneimine) may have average molecular weight of about 1 kD to 50 kD, specifically about 10 kD to 25 kD, but is not limited thereto. By further comprising the cationic material, particle size may be reduced when forming a complex, endosomal escape of nucleic acid may be assisted, thus increasing nucleic acid delivery efficiency.

The cationic material may be low density lipoprotein-like (LDL-like) nanoparticle of a core-shell structure comprising a core comprising cholesteryl ester and triglyceride; and a shell comprising cholesterol, fusogenic lipid, cationic lipid, and a lipid-PEG (polyethyleneglycol) conjugate.

The cholesteryl ester refers to a compound wherein C10-24 saturated or unsaturated fatty acid is ester-bonded to cholesterol. Preferably, it may be ester of C16-18 unsaturated fatty acid such as oleic acid.

The triglyceride may be purified triglyceride having a composition of various fatty acids, or vegetable oil having triglyceride consisting of multiple fatty acids as a main ingredient. Specifically, the triglyceride may be animal or vegetable oil, and it may be at least one selected from the group consisting of soybean oil, olive oil, cotton seed oil, sesame oil, liver oil, and the like. As the oil, one kind may be used or many kinds of oils may be mixed and used. According to specific example, the triglyceride may be triolein.

The cholesteryl ester and triglyceride may form a core of the low density lipoprotein-like nanoparticle through a hydrophobic bond.

The fusogenic lipid may include all kinds of neutral, cationic or aionic lipid that can form the nanoparticles, and it may be a mixture of single kind or many kinds of phospholipids. The fusogenic lipid may include all kinds of phospholipid that can induce fusion, and for example, it may be phosphatidylcholin (PC). phosphatidylethanolamine, phosphatidylserine, phosphatidylglycerol, or lysoforms thereof, or completely saturated or partially cured forms having a C6-24 aliphatic chain. Specifically, the fusogenic lipid may be at least one selected from the group consisting of dioleoylphosphatidylethanolamine (DOPE), palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), distearoylphosphatidylethanolamine (DSPE), phosphatidylethanolamine (PE), dipalmitoylphosphatidylethanolamine, 1,2-dioleoyl-sn-glycero-3-phosphoethanolamine, 1-palmitoyl-2-oleoyl-sn-glycero-3-phosphoethanolamine (POPE), 1-palmitoyl-2-oleoyl-sn-glycero-3-phosphocholine (POPC), 1,2-dioleoyl-sn-glycero-3-[phospho-L-serine] (DOPS), 1,2-dioleoyl-sn-glycero-3-[phospho-L-serine], and the like, but is not limited thereto.

The fusogenic lipid and cholesterol that constitute a shell may improve transfection efficiency, and performs a function as helper lipid for decreasing cytotoxicity of combined cationic lipid. And, the cholesterol affords stiffness in terms of shape, thus improving activity of helper and stability of nanoparticles. And, fusogenic lipid assists in passage over cell membrane and endosomal escape of nanoparticles to facilitate intracellular delivery.

The cationic lipid includes those having net negative charge at specific pH such as physiological pH. Specifically, the cationic lipid may be at least one selected from the group consisting of 3beta-[N—(N′,N′,N′-trimethylaminoethane)carbamoyl]cholesterol (TC-cholesterol), 3beta[N—(N′,N′-dimethylaminoethane)carbamoyl]cholesterol (DC-cholesterol), 3beta[N—(N′-monomethylaminoethane)carbamoyl]cholesterol (MC-cholesterol), 3beta[N-(aminoethane)carbamoyl]cholesterol (AC-cholesterol), N—(N′-aminoethane)carbamoylpropanoic tocopherol (AC-tocopherol), N—(N′-methylaminoethane)carbamoylpropanoic tocopherol (MC-tocopherol), N,N-dioleyl-N,N-dimethylammoniumchloride (DODAC), N,N-distearyl-N,N-dimethylammoniumbromide (DDAB), N-(1-(2,3-dioleyloxy)propyl-N,N,N-trimethylammoniumchloride (DOTAP), N,N-dimethyl-(2,3-dioleyloxy)propylamine (DODMA), N-(1-(2,3-dioleyl)propyl)-N,N,N-trimethylammonium chloride (DOTMA), 1,2-dioleyl-3-dimethylammonium-propane (DODAP), 1,2-dioleylcarbamyl-3-dimethylammonium-propane (DOCDAP), dilineoyl-3-Dimethylammonium-propane (DLINDAP), dioleyloxy-N-[2-sperminecarboxamido]ethyl}-N,N-dimethyl-1-propanaminum trifluoroacetate (DOSPA), dioctadecylamidoglycyl spermine (DOGS), 1,2-dimyristyloxypropyl-3-dimethyl-hydroxyethyl ammonium bromide (DMRIE), 3-dimethylamino-2-(cholest-5-en-3-beta-oxybutane-4-oxy)-1-(cis,cis-9,12-octadecadienoxy)propane (CLinDMA), 2[5′-(cholest-5-en-3-beta-oxy)-3′-oxapentoxy]-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-diacyl-3-trimethylammonium-propane (TAP), 1,2-diacyl-3-dimethylammonium-propane (DAP), 1,2-di-O-octadeceyl-3-trimethylammonium propane, 1,2-dioleoyl-3-trimethylammonium propane, Trasfectam®, 98N12-5(1), and the like.

Particularly, DC-cholesterol has lower toxicity than other cationic lipids, and gene carriers of DC-cholesterol type have been approved for use in clinical treatment of various diseases including melanoma, cystofibroma, uterine cervical cancer, breast cancer and ovarian cancer, and the like, and thus, DC-cholesterol may be preferably used.

The lipid-PEG (polyethyleneglycol) conjugate refers to a form wherein lipid and PEG are conjugated.

Lipid in the conjugate may be selected from the group consisting of all kinds of cholesterols as previously described, fusogenic lipid and cationic lipid, and for example, it may be lipid including an amine group. According to specific embodiment, the lipid may be at least one selected from the group consisting of cholesterol, dioleoylphosphatidylethanolamine (DOPE), palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), distearoylphosphatidylethanolamine (DSPE), phosphatidylethanolamine (PE), dipalmitoylphosphatidylethanolamine (DPPE), 1,2-dioleoyl-sn-glycero-3-phosphoethanolamine, 1-palmitoyl-2-oleoyl-sn-glycero-3-phosphoethanolamine (POPE), 1-palmitoyl-2-oleoyl-sn-glycero-3-phosphocholine (POPC), 1,2-dioleoyl-sn-glycero-3-[phospho-L-serine] (DOPS), 1,2-dioleoyl-sn-glycero-3-[phospho-L-serine], 3beta-[N—(N′,N′,N′-trimethylaminoethane)carbamoyl]cholesterol (TC-cholesterol), 3beta-[N—(N′,N′- [symbol] methylaminoethane)carbamoyl]cholesterol (DC-cholesterol), 3beta[N—(N′-monomethylaminoethane)carbamoyl]cholesterol (MC-cholesterol), 3beta[N—(aminoethane)carbamoyl]cholesterol (AC-cholesterol), N—(N′-aminoethane)carbamoylpropanoic tocopherol (AC-tocopherol), N—(N′-methylaminoethane)carbamoylpropanoic tocopherol (MC-tocopherol), N,N-dioleyl-N,N-dimethylammoniumchloride (DODAC), N,N-distearyl-N,N-dimethylammoniumbromide (DDAB), N-(1-(2,3-dioleyloxy)propyl-N,N,N-trimethylammoniumchloride (DOTAP), N,N-dimethyl-(2,3-dioleyloxy)propylamine (DODMA), N-(1-(2,3-dioleyl)propyl)-N,N,N-trimethylammonium chloride (DOTMA), 1,2-dioleyl-3-dimethylammonium-propane (DODAP), 1,2-dioleylcarbamyl-3-dimethylammonium-propane (DOCDAP), dilineoyl-3-Dimethylammonium-propane (DLINDAP), dioleyloxy-N-[2-sperminecarboxamido)ethyl}-N,N-dimethyl-1-propanaminiumtrifluoroacetate (DOSPA), diocadecylamidoglycyl spermine (DOGS), 1,2-dimyristyloxypropyl-3-dimethyl-hydroxyethyl ammonium bromide (DMRIE), 3-dimethylamino-2-(cholest-5-en-3-beta-oxybutane-4-oxy)-1-(cis,cis-9,12-octadecadienoxy)propane (CLinDMA), 2-[5′-(cholest-5-en-3-beta-oxy)-3′-oxapentoxy]-3-dimethyl-1-(cis,cis-9′,12′-otcadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-diacyl-3-trimethylammonium-propane (TAP), 1,2-diacyl-3-dimethylammonium-propane (DAP), 1,2-di-O-octadeceyl-3-trimethylammonium propane, 1,2-dioleoyl-3-trimethylammonium propane, Trasfectam®, 98N12-5(1), and the like. Specifically, the lipid may be distearoylphosphatidylethanolamine (DSPE), dipalmitoylphosphatidylethanolamine (DPPE), and the like, but is not limited thereto.

The PEG may have average molecular weight of 100 to 100,000 Daltons, specifically 500 to 70,000 Daltons, more specifically 1,000 to 50,000 Daltons, but it not limited thereto. According to one embodiment, the PEG may be functionalized PEG at a part where it is not bonded to lipid, wherein the functional group that can be used may at least one selected from the group consisting of succinyl, carboxylic acid, maleimide, amine, biotin, cyanur, folate, and the like.

The mole ratio of lipid and PEG in the lipid-PEG conjugate may be about 1:0.5 to 3 (lipid moles:PEG moles).

According to an embodiment, the lipid-PEG conjugate may be distearoylphosphatidylethanolamine (DSPE)-PEG, DPPE-PEG, and the like. The lipid-PEG conjugate may contribute to particle stability in serum, and performs a function for protecting nucleic acid from decomposition enzyme during in vivo delivery, thus increasing in vivo stability of nucleic acid.

According to an embodiment, the nanoparticle may be low density lipoprotein-like nanoparticle comprising a core comprising cholesteryl oleate and triolein; and a shell comprising cholesterol, dioleoylphosphatidylethanoleamine (DOPE), 3β[N—(N′,N′-dimethylaminoethane)carbamoyl]-cholesterol (DC-cholesterol), and DSPE-PEG.

The cationic lipid located in the shell of the nanoparticle may be bonded to drugs, particularly, anionic drugs and/or nucleic acid through electrostatic bonding to form a conjugate.

The low density lipoprotein-like nanoparticle may comprise 30 to 60 wt % of cholesteryl ester, 0.1 to 10 wt % of triglyceride, 5 to 20 wt % of cholesterol, 5 to 30 wt % of fusogenic lipid, 10 to 50 wt % of cationic lipid, and 0.01 to 1 wt % of a lipid-PEG conjugate. Preferably, it may comprise 40 to 50 wt % of cholesteryl ester, 1 to 5 wt % of triglyceride, 8 to 12 wt % of cholesterol, 12 to 1 wt % of fusogenic lipid, 25 to 30 wt % of cationic lipid, and 0.05 to 0.5 wt % of lipid-PEG conjugate.

If the content of lipid-PEG conjugate in the nanoparticle is greater than the above range, it may be difficult to form a complex with nucleic acid, and if it is less than the above range, it may be unfavorable in terms of serum stability of particle and in vivo stability of nucleic acid, and thus, considering easiness of complex formation of nanoparticles and nucleic acid, in vivo stability of nanoparticles, in vivo stability of nucleic acid, and the like, the content of lipid-PEG conjugate may be preferably within the above range.

And, the weight ratio of the core and the shell in the nanoparticle may be 30:70 to 70:30, specifically 40:60 to 60:40, more specifically 45:55 to 55:45.

The nanoparticles may have average particle diameter of 70 nm to 110 nm so as to facilitate introduction into liver cells.

According to an embodiment, the lipoprotein-like nanoparticle may be hydrophobically surface-modified particle, and for example, it may further comprise at least one selected from the group consisting of hydrophobically surface-modified Quantum Dot, Carbone Dot, gold nanoparticles, iron oxide nanoparticles, and the like. As such, if the lipoprotein-like nanoparticle further comprise hydrophobically surface-modified particle, it may be advantageously applied in the field of diagnosis because imaging may become easy. Wherein, the hydrophobically surface-modified particles may have average diameter of 2 nm to 50 nm, for example, 2 nm to 20 nm, but is not limited thereto. The content of the hydrophobically surface-modified particles may be 1% (w/w) to 20% (w/w), based on the total weight of nanoparticles (the weight of nanoparticles including hydrophobically surface-modified particles).

According to another embodiment, in the shell of the lipoprotein-like nanoparticle, at least one of the constituting elements of the shell, namely, cholesterol, fusogenic lipid, cationic lipid and lipid-PEG conjugate, may be labeled with radio isotope. As such, if the shell of the nanoparticle further comprises radio isotope labeled lipid, it may be more advantageously applied in the field of diagnosis because imaging of nanoparticles may become easy. Wherein, the radio isotope that can be used may be selected from the group consisting of P32, C11, H3, O15, N13, and the like, and the amount of radio isotope labeled lipids that are further included may be 0.001 to 5% (w/w), based on the total weight of lipoprotein-like nanoparticles (the weight of nanoparticles comprising radio isotope labeled lipids). When the radio isotope is labeled at a lipid-PEG conjugate, it may be labeled at a functional group that can be bonded to lipid or a functional group of PEG. Wherein, even after adding the radio isotope labeled lipids, the added amount of the radio isotope labeled lipids may be controlled within the above range so that the ratio of the shell and the core may be within the above described range.

The hyaluronic acid-alkylenediamine-nucleic acid complex may achieve the following advantages by conjugating hyaluronic acid to nucleic acid through a disulfide bond. Nucleic acid, particularly, siRNA is unfavorable when forming a conjugate with cationic material due to its weak anionic property and firm structure, but by conjugating hyaluronic acid with strong anionic property, it may form a conjugate more easily. And, hyaluronic acid performs a function for protecting nucleic acid (for example, siRNA) from an attack by nucleic acid decomposition enzyme (for example, RNase) in vivo or in the intracellular environment, thus increasing in vivo stability of the nucleic acid conjugate. And, if it further comprise cationic material such as linear polyethyleneimine, and the like, surface charge may be lowered, thus decreasing non-specific in vivo interaction.

Another embodiment relates to a composition for nucleic acid delivery, comprising the hyaluronic acid-alkylenediamine-nucleic acid complex as an active ingredient. As described above, since the hyaluronic acid-alkylenediamine-nucleic acid complex comprises anionic polymer with excellent biocompatibility, i.e., hyaluronic acid, high positive charge of alkylenediamine may be neutralized by increasing anionic property, and thus, intracellular and in vivo toxicity may be decreased and in vivo delivery efficiency may be increased. And, since the conjugate has a form wherein nucleic acid is connected to the conjugate through a disulfide bond instead of a simply mixed form of nucleic acid with hyaluronic acid-alkylenediamine conjugate, it may be more favorable for formation of a conjugate, nucleic acid decomposition by nucleic acid decomposition enzyme may be decreased to increase stability, and non-specific in vivo interactions may be decreased.

The composition for delivery of nucleic acid may be administered to mammals, for example, human, and it may be appropriately administered by intravenous, intramuscular, subcutaneous, oral, intrabone, transdermal, local tissue routes, and the like, according to administration purpose, and it may be appropriately formulated in various forms such as a solution, a suspension injection, a tablet or a capsule, and the like, according to administration form, administration purpose, administration subject, and the like.

Still another embodiment relates to a method for preparing the hyaluronic acid-alkylenediamine conjugate. Specifically, the method may comprise bonding one amine group of the two amine groups of alkylenediamine to the carboxylic acid group of hyaluronic acid to form a hyaluronic acid-alkylenediamine conjugate.

Still another embodiment relates to a method for preparing the hyaluronic acid-alkylenediamine-nucleic acid complex.

Specifically, the method may comprise

bonding one amine group of the two amine groups of alkylenediamine to the carboxylic acid group of hyaluronic acid to form a hyaluronic acid-alkylenediamine conjugate;

preparing nucleic acid with a thiol group introduced at the 3′ end; and

forming a disulfide bond between the other amine group of the above formed hyaluronic acid-alkylenediamine conjugate and the thiol group introduced at the 3′ end of the nucleic acid, to produce a hyaluronic acid-alkylenediamine-nucleic acid complex.

The step of forming the hyaluronic acid-alkylenediamine conjugate may comprise, for example, adding 1-ethyl-3-[3-(dimethylamino)propyl]carbodiimide (EDC), and at least one compound selected from the group consisting of 1-hydroxy bentriazole monohydrate (HOBt), N-hydroxysulfosuccinimide (sulfo-NHS) and N-hydroxysuccinimide (NHS) to hyaluronic acid, to bond alkylenediamine to the carboxylic acid group of hyaluronic acid (HA), thus forming a hyaluronic acid-alkylenediamine conjugate. Wherein, the added amounts of the at least one selected HOBt, sulfo-NHS and NHS, and EDC may be respectively 1:4 to 1:20, based on the equivalent ratio compared to the carboxylic acid groups of hyaluronic acid (equivalent of the carboxylic acid of hyaluronic acid: equivalent of at least one selected from the group consisting of HOBt, sulfo-NHS, and NHS, or equivalent of EDC).

As explained above, since the formation process of the hyaluronic acid-alkylenediamine conjugate may control substitution rate by controlling the reaction time, a conjugate with a desired substitution rate may be easily prepared. For example, if a conjugate with high substitution rate is to be produced, the reaction time may be controlled long, and if a conjugate with low substitution rate is to be produced, the reaction time may be controlled short. For example, the reaction time may be controlled within about 3 minutes to about 48 hours, and after the reaction, pH may be controlled using a NaOH 1M aqueous solution, and then, unreacted samples may be removed through filtration to obtain appropriate substitution rate.

The step of preparing the thiol group-introduced nucleic acid may be conducted by introducing a thiol group at the 3′ end of nucleic acid by any method commonly known in the art or acquiring thiol group-introduced nucleic acid, and it may be conducted before, after or simultaneously with the step of forming the hyaluronic acid-alkylenediamine conjugate.

In the step of producing the hyaluronic acid-alkylenediamine-nucleic acid complex, Succinimidyl 3-(2-pyridyldithio)propionate (SPDP) is added to the amine group of the above synthesized hyaluronic acid-alkylenediamine conjugate (for example, HA-DAB) and the thiol group-introduced nucleic acid (for example, siRNA with a thiol group introduced at the 3′ end of sense strand) in the amount of 1 to 3 moles, specifically about 2 moles of the amine group of the hyaluronic acid-alkylenediamine conjugate, and reacted to introduce a pyridyl group, and the amine group of the hyaluronic acid-alkylenediamine conjugate and the thiol group of nucleic acid are bonded through a thiol-exchange reaction, thus producing a hyaluronic acid-alkylenediamine-nucleic acid complex including a disulfide bond.

As explained above, if a hyaluronic acid-nucleic acid conjugate is formed through alkylenediamine, the production rate of the conjugate may be about 60% or more, for example, about 60 to 70%, thus achieving remarkably excellent conjugate production rate, compared to about 20% or less conjugate production rate of the existing method without using alkylenediamine.

The method may further comprise a step of adding linear polyethyleneimine (linear PEI: for example, average molecular weight 10 kD to 25 kD) that is low toxic cationic material to the produced hyaluronic acid-alkylenediamine-nucleic acid complex.

Still another embodiment relates to a method for in vivo delivery of nucleic acid, comprising administering the hyaluronic acid-alkylenediamine-nucleic acid complex to a subject.

The hyaluronic acid-alkylenediamine-nucleic acid complex may further comprise cationic material, which is explained above.

The subject may be mammals, for example, human, and it may be a subject in need of administration of nucleic acid included in the hyaluronic acid-alkylenediamine-nucleic acid complex. The administration route may include intravenous, intramuscular, subcutaneous, oral, intrabone, transdermal, local tissue administration, and the like, but is not limited thereto.

According to the present invention, hyaluronic acid with negative charge and excellent biocompatibility is introduced in nucleic acid, to improve in vivo stability of nucleic acid, and further facilitate the formation of a conjugate with cationic material, thereby improving nucleic acid delivery efficiency. And, since hyaluronic acid has liver targetability when in vivo delivered, it may be used for the development of liver targeted nucleic acid delivery system.

The present invention is further explained in more detail with reference to the following examples. These examples, however, should not be interpreted as limiting the scope of the present invention in any manner.

### Example 1

### Synthesis of HA-siRNA by Disulfide Bonding of HA and siRNA

### 1-1: Synthesis of HA-DAB

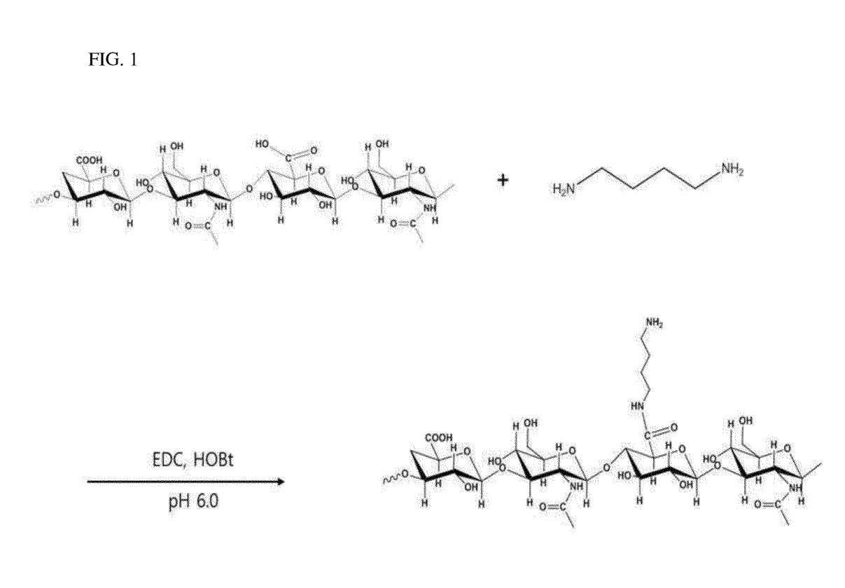

A method of synthesizing HA-DAB (1,4-diaminobutane) for the development of a HA-siRNA conjugate is schematically shown in FIG. 1. Specifically, the carboxyl group of HA (100 g) with molecular weight of 100 kDa was activated with 1-ethyl-3-[3-(dimethylamino)-propyl]carbodiimide (EDC) and 1-hydroxy bentriazole monohydrate (HOBt) at pH 6.0, and then, 1,4-diaminobutane (DAB) was added to synthesize HA-DAB. The EDC, HOBt, and DAB were added respectively in the amount of 4, 4 and 10 times compared to the HA unit (M.W. 401) (based on mole). The DAB was treated in the excessive amount of 4 moles or more based on the carboxyl group of HA so that crosslink between HAs may not occur.

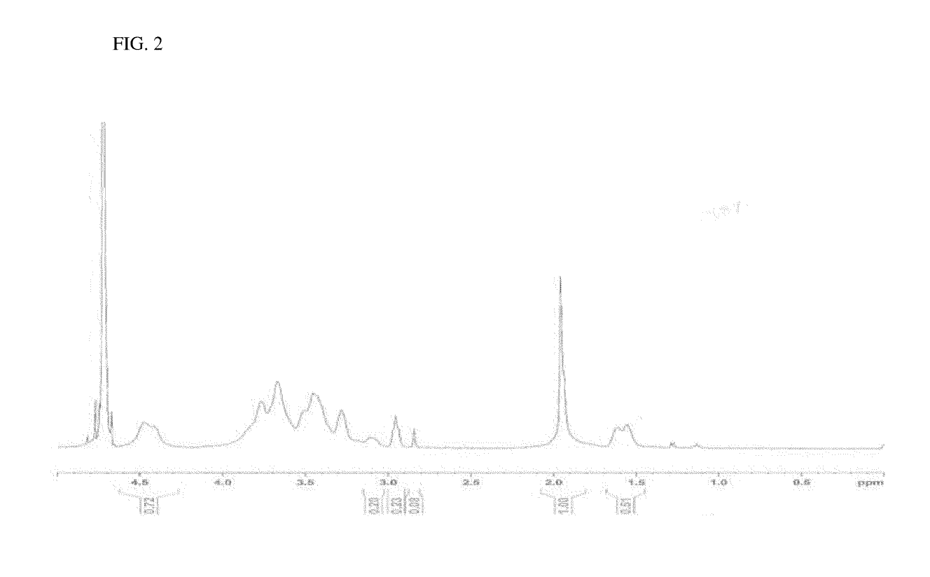

The reaction time was varied to 1 hour, 6 hours, and 24 hours to obtain synthetic products with different substitution rates. The obtained synthetic product was filtered and purified using a filtration tube (cut off 10 kDa) to prepare HA-DAB. The synthesis of HA-DAB was confirmed by 1H NMR and the result is described in FIG. 2. As shown in FIG. 2, from the 1H NMR result, the substitution rate of HA-DBA (measuring after adjusting pH to 7 using a NaOH 1M aqueous solution) may be calculated by comparing HA peak area (1.9 ppm) and DAB peak area (1.6 ppm). The substitution rates were respectively 19.2%, 37.8% and 43.4% when reacted for 1 hour, 6 hours and 24 hours.

### 1-2: Synthesis of Disulfide Bond-Introduced HA-DAB-siRNA

Through the process as shown in FIG. 3, a disulfide bond-introduced HA-siRNA conjugate was synthesized. Specifically, the HA-DAB (20 mg; 4.8×10⁻⁵ moles) synthesized in Example 1-1 and Succinimidyl 3-(2-pyridyldithio)propionate (SPDP) (5.6 mg; 1.8×10⁻⁵ moles) were mixed and reacted to introduce a pyridyl group in the HA chain, and the mixture was filtered and purified using a filtration tube (M.W.C.O 100 kDa). The purified HA-DAB-SPDP was treated with TCEP (tris(2-carboxyethyl)phosphine), and absorbance was measured at 343 nm to calculate the concentration of pyridyl groups (the concentration of pyrimidyl groups: 34 mM).

To the quantified HA-DAB-SPDP solution, anti-luciferase siRNA with thiol groups introduced at the 3′ end of sense strand (sense strand: CCACACUAUUUAGCUUCUUdTdT (SEQ ID NO. 1-dTdT); antisense strand: AAGAAGCUAAAUAGUGUGGdTdTdT (SEQ ID NO. 2-dTdTdT)) was added at various ratios of 0.2 moles to 1 mole, based on the concentration of the pyrimidyl groups. In the synthesized HA-DAB-siRNA, the number of siRNA conjugated per one molecule of HA 100 kDa may be controlled to 2 to 15.

### Experimental Example 1

### Confirmation of HA-DAB-siRNA Complex

High performance liquid chromatography (HPLC) was used to confirm the production of the HA-DAB-siRNA complex synthesized in Example 1-2. Wherein, a superdex 200 10/300 GL (GE health) column was used, pH 7.0 50 mM phosphate buffer was used as a mobile phase, flow rate was 0.5 ml/min, and absorbance was measured at 210 nm and 260 nm. The result is shown in FIG. 4a, and synthetic product was separated and purified based on the result. As shown in FIG. 4a, the production rate (reaction rate) of the HA-DAB-siRNA complex was confirmed to be about 64%. Meanwhile, the result of measuring absorbance of a HA-siRNA conjugate synthesized using 3-(2-pyridyldithio)propionyl hydrazide (PDPH) instead of SPDP without using DAB at 260 nm is shown in FIG. 4b. As calculated from FIG. 4b, the production rate of the HA-siRNA conjugate was about 12%.

The production rate (reaction rate) of the conjugate was calculated by the following Equation:

Reaction rate(%)={(the amount of siRNA consumed for the production of a conjugate)/(the amount of siRNA added at the beginning of the reaction)}×100

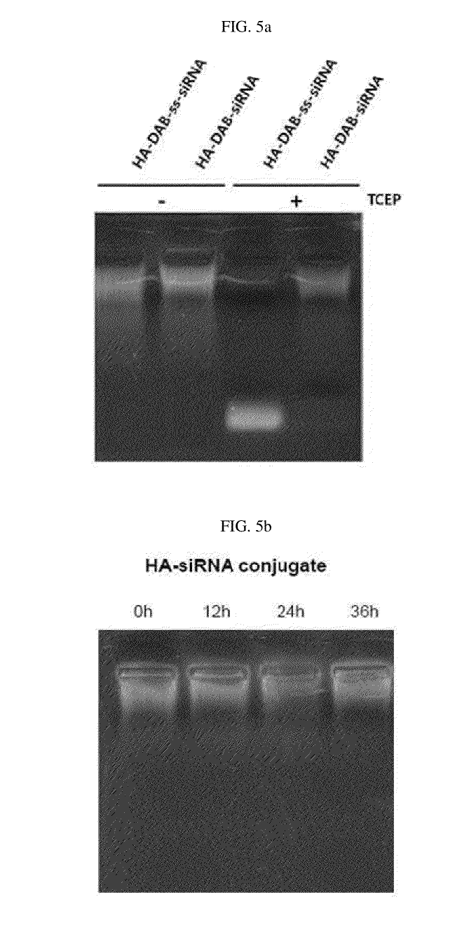

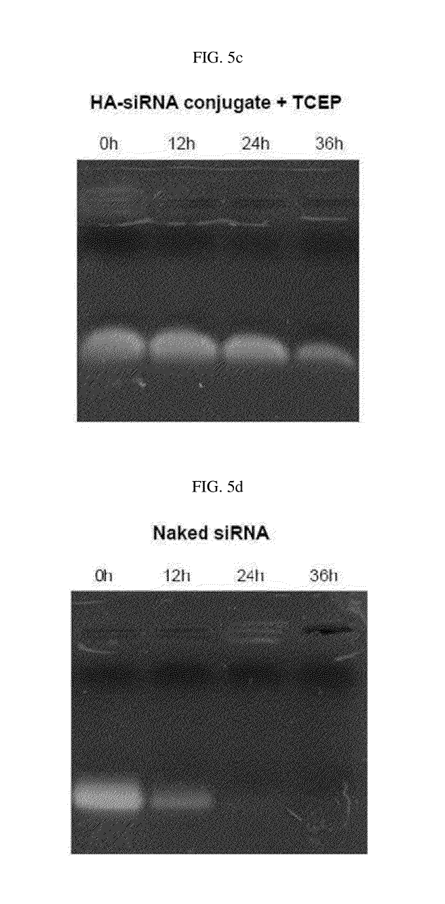

And, the synthesis and the decomposition of the separated and purified synthetic product were confirmed by agarose gel electrophoresis, and the results are shown in FIGS. 5a to 5d.

FIGS. 5a to 5d show the results of analysis of HA-DAB-siRNA complexes synthesized using different two cross linkers by agarose electrophoresis. Specifically, in FIG. 5a, HA-ss-siRNA denotes a conjugate including a disulfide bond (—ss) between HA and siRNA, and HA-siRNA denotes a conjugate connected by a common covalent bond instead of a disulfide bond (control). As shown in the two right columns, when treating a reducing agent TCEP, siRNA is separated and a band appears at the bottom in the conjugate including a disulfide bond, while siRNA is not separated and remains at the top in the control.

And, to evaluate the stability of the HA-DAB-siRNA complex, FBS was added to the HA-DAB-siRNA complex and the remaining amount of siRNA was confirmed by agarose electrophoresis according to time (in FIGS. 5b and 5c, a HA-siRNA conjugate denotes a HA-DAB-siRNA complex). As the result, it was confirmed that the HA-DAB-siRNA complex was not decomposed and remained for a long time compared to non-conjugated siRNA (naked siRNA).

### Experimental Example 2

### Evaluation of Properties of HA-DAB-siRNA/IPEI Particles

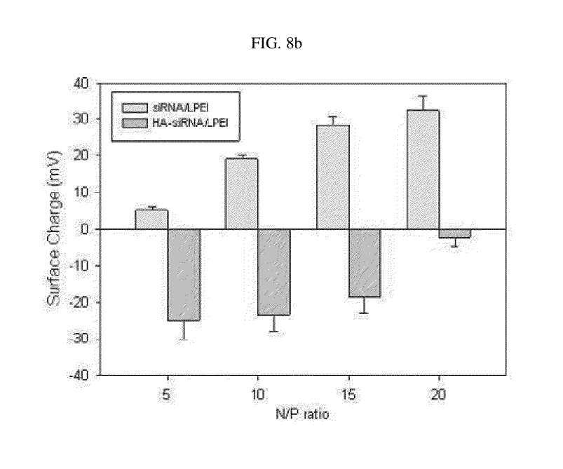

To evaluate the properties of HA-DAB-siRNA/IPEI particles, the above prepared HA-DAB-siRNA (the amount corresponding to 1 nmol based on siRNA) and various N/P ratios of IPEI (linear polyethyleneimine; molecular weight 25 kDa) were mixed to form a complex, it was diluted in PB2 1 ml, the particle size and the surface charge were measured using a DLS analyzer (Zetasizer Nano, Malvern Instrument Co., UK), and the results are respectively shown in FIGS. 8a (particle size) and 8b (surface charge).

FIG. 8a shows the result of measuring the particle size when forming complexes with IPEI at various N/P ratios, wherein in the case of a siRNA/IPEI complex, a dense complex is not formed and the size is large, while in the case of a HA-DAB-siRNA/IPEI complex, a dense complex is formed due to negative charge property of HA and structural flexibility, and thus the size is observed relatively small. FIG. 8b shows the result of measuring the surface charge at various N/P ratios, and it is anticipated that since siRNA/IPEI is strongly positively charge, delivery efficiency may be decreased due to non-specific in vivo interactions with blood ingredients, and high toxicity may be exhibited, while HA-DAB-siRNA/IPEI is negatively charged thus overcoming these problems.

### Experimental Example 3

### Evaluation of Cytotoxicity and Gene Inhibition Efficiency of HA-DAB-siRNA

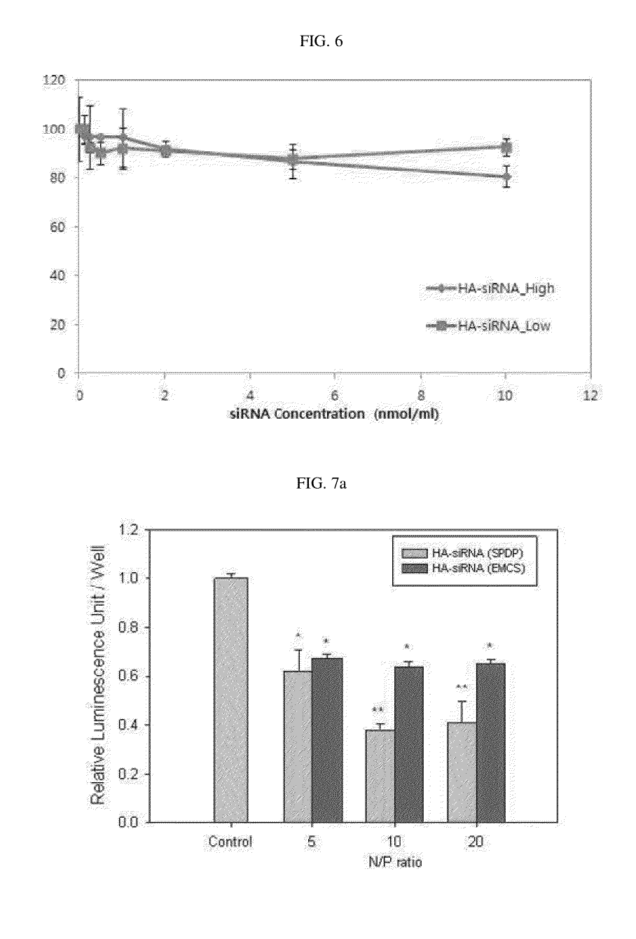

To evaluate the toxicity of a HA-DAB-siRNA complex, MTT assay was conducted. Specifically, MDA-MB-231 cells (CELL BIOLABS, INC, CA, US/ESM000113) that had been cultured in DMEM medium (GIBCO-BRL, NY, USA) containing 10% (v/v) fetal bovine serum and 100 U/Ml penicillin-streptomycin were seeded in a 96-well plate at 5×10³ cells/well, and cultured at 37° C. and 5% CO₂ until the cells grow about 70%, and then, the HA-DAB-siRNA prepared in Example 1-2 was injected into each well at various concentrations and cultured for 24 hours. And then, cytotoxicity was evaluated by MTT assay and the results (cell viability) are shown in FIG. 6.

In FIG. 6, the expression ‘HA-DAB-siRNA_High’denotes those having substitution rate of 38%, and the expression ‘HA-DAB-siRNA_Low’ denotes those having substitution rate of 19%. As confirmed in FIG. 6, the HA-DAB-siRNA complex does not exhibit cytotoxicity regardless of DAB substitution rate.

To evaluate gene inhibition efficiency of a HA-DAB-siRNA complex, a complex was formed with linear polyethyleneimine with low toxicity, the cells were treated therewith, and the degree of gene expression was measured. Specifically, luciferase expressing MDA-MB-231 cells (CELL BIOLABS, INC, CA, US/ESM000113) that had been cultured in DMEM medium (GIBCO-BRL, NY, USA) containing 10% fetal bovine serum and 100 U/mL penicillin-streptomycin were seeded into a 96-well plate at 5×10³ cells/well, and cultured at 37° C. and 5% CO₂ until the cells grow about 70%.

To form a complex, the HA-DAB-siRNA complex prepared in Example 1-2 and linear polyethyleneimine (25 Kd) were mixed at various ratios in a 150 mM NaCl solution for 15 minutes, and stored. And then, the previously prepared cells were treated therewith, and additionally cultured for 24 hours. After 24 hours, the cells were treated with cell lysis buffer (4M NaCl, 1M Tris-Cl (pH 7.5), 0.5M EDTA (pH 8.0), 1M NaF, 500 mM NaVO3, 1M DTT, 100 mM PMSF/(Bio Prince Korea Co., Ltd)), and luciferase activity was measured for 30 seconds using Luinometer, and the results are shown in FIGS. 7a and 7b.

In FIGS. 7a and 7b, control denotes a group that was not treated with siRNA. In FIG. 7a, N/P ratio denotes the mole number of amine groups of liner PEI compared to phosphate of siRNA, HA-siRNA (SPDP) is a conjugate wherein siRNA is connected by a disulfide bond through a linker SPDP (see FIG. 3: at one side is a pyridyl group, and at the other side is a NHS group), and HA-siRNA (EMCS; N-epsilon-Malemidocaproyl-oxysuccinimide ester) is a control conjugate without a disulfide bond, prepared so as not to be dissociated in the cells using a linker that does not include a disulfide bond (at one side is an NHS group, and at the other side is a maleimide group, thus connecting an amine group and a thiol group).

As confirmed in FIG. 7a, disulfide bond-introduced HA-siRNA (SPDP) has excellent luciferase inhibition efficiency.

FIG. 7b shows the result of experiment observing and comparing gene inhibition efficiencies while varying the number of siRNA bonded per one HA chain. In FIG. 7b, HA-siRNA_{—}2, HA-siRNA_{—}7, HA-siRNA_{—}11 respectively denote that the number of siRNA is 2, 7 and 11, and siRNA/Lipofectamine (Lipo) denotes positive control formed of siRNA and Lipofectamine 2000 (Invitrogen) together and treated under the same conditions as the above experiment. As can be seen from FIG. 7b, all the tested complexes have excellent luciferase inhibition efficiencies, and particularly, when using HA-siRNA_{—}7 with 7 siRNAs bonded to one HA chain, gene inhibition efficiency was observed high.

### Experimental Example 4

### Confirmation of Cellular Delivery Mechanism of HA-DAB-siRNA/IPEI Particles

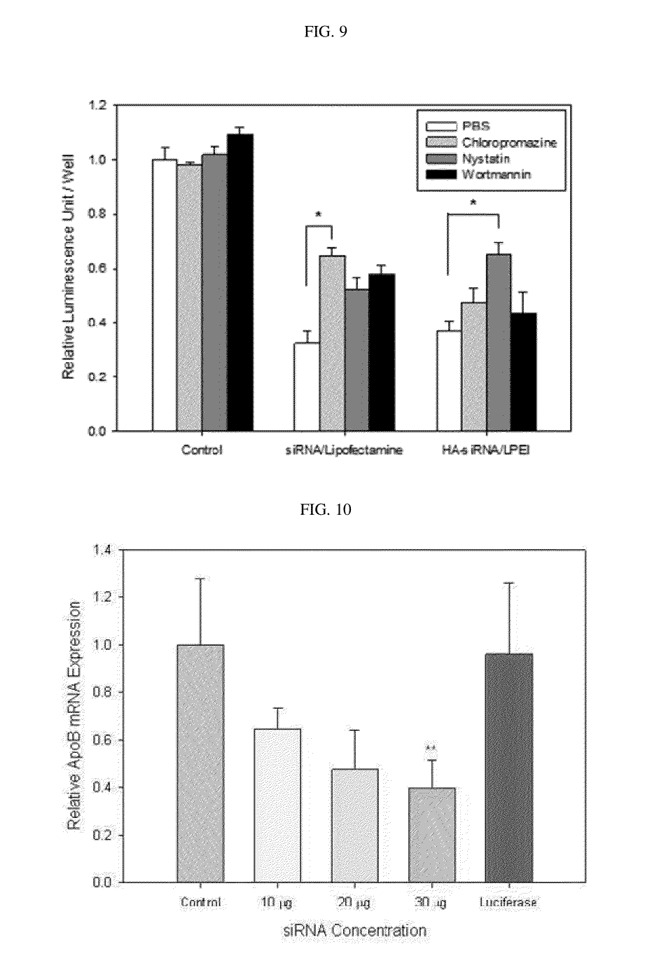

To confirm the cellular delivery mechanism of HA-DAB-siRNA/IPEI particles, a HA-DAB-siRNA complex with linear polyethyleneimine was prepared by the same method as Experimental Example 3, except using siRNA labeled with cy3 at the 5′ end of sense strand, purchased from bioneer corporation. The cells were treated therewith, and various cellular uptake inhibitors were administered. More specifically, 2×10⁵ MDA-MB-231 cells (CELL BIOLABS, INC, CA, US/ESM000113) were grown in 8 chamber slide glass for one day, the medium was replaced with those to which Chloropromazine (10 μg/mL; Sigma Aldrich), Nystatin (50 μg/mL; Sigma Aldrich), wortmannin (50 μg/mL; Sigma Aldrich) were respectively added (DMEM medium containing 10% fetal bovine serum and 100 U/mL penicillin-streptomycin (GIBCO-BRL, NY, USA) as cellular uptake inhibitor, and then, the cells were treated with a HA-DAB-siRNA/LPEI complex (0.5 nmol/ml based on the concentration of siRNA) and incubated for 2 hours. The medium containing each inhibitor was replaced, and then, luciferase activity was confirmed by the method of Experimental Example 3, and the results are shown in FIG. 9.

As shown in FIG. 9, it was confirmed that cellular delivery is inhibited most by nystatin which inhibits clathrine mediated endocytosis. This result corresponds to the previously known cellular delivery mechanism of HA, and shows that HA-DAB-siRNA/IPEI is delivered into the cells by the same mechanism as HA.

### Experimental Example 5

### Confirmation of Gene Inhibition Effect of HA-DAB-siRNA/IPEI Particles in Mouse Liver Tissue

To evaluate in vivo gene inhibition effect of a HA-DAB-siNRA/Lpei complex, a complex of HA-DAB-siRNA that was prepared by the same method as Example 1-2 except using the following siRNA against Apolipoprotein B, and linear PEI was formed, and then, it was administered to tail vein of mouse (balb/c mouse, 6 week-aged, body weight 25 g; Postech, Animal Lab.) at various administration amounts.

siNRA Against Apolipoprotein B

sense strand;
(SEQ ID NO. 3-dTdT)
5′-GUC AUC ACDA CUG AAU ACC AAU dTdT-3′
antisense strand:
(SEQ ID NO. 4)
5′-AUU GGU AUU CAG UGU GAU GAC-3′

After one day, liver tissue was extracted to separated mRNA, RT-PCR was conducted using the following primer, and the result of quantitative analysis is shown in FIG. 10:

sense primer for ApoB:
(SEQ ID NO. 5)
5′-TTT TCC TCC CAG ATT TCA AGG-3′
antisense primer for ApoB:
(SEQ ID NO. 6)
5′-TCC AGC ATT GGT ATT CAG TGT G-3′
sense primer for GAPDH:
(SEQ ID NO. 7)
5′-CCT TCA TTG ACC TCA ACT AC-3′
antisense primer for GAPDH:
(SEQ ID NO. 8)
5′-GGA AGG CCA TGC CAG TGA GC-3′

PCR conditions: 40 cycles of denaturation at 95° C. for 5 minutes, and then, 95° C. 30 seconds and 53 r 30 seconds

As shown in FIG. 10, it was confirmed that the amount of mRNA of target gene (Apolipoprotein B) in liver tissue decreases in proportion to the amount of injected siRNA. As control, non-specific luciferase siRNA (prepared in Example 1-2) was used.

### Experimental Example 6

### Evaluation of Properties of HA-DAB-siRNA/CSLN Complex

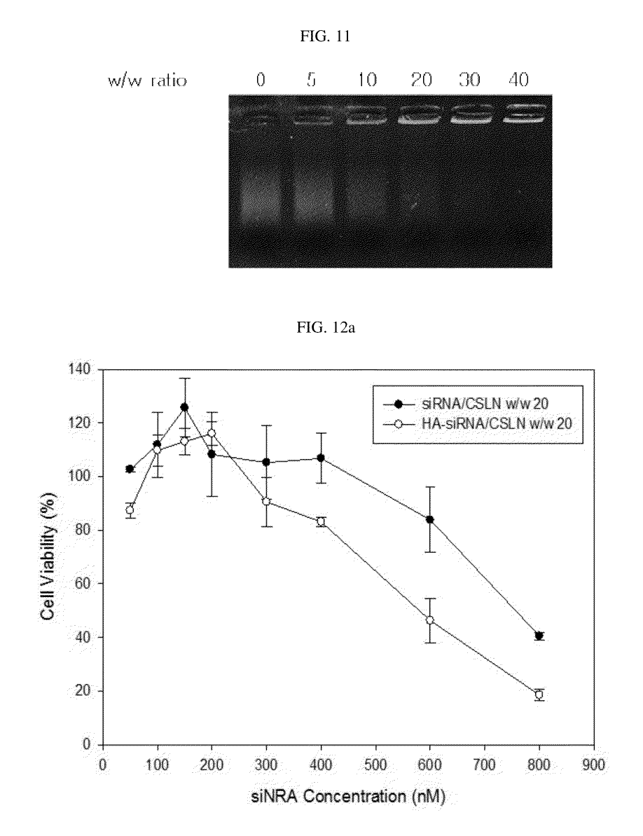

CSLN(Cationic Solid Lipid Nanoparticle) that consists of Cholesterol oleate (22.5 mg, 45.0% w/w), triolein (1.5 mg, 3.0% w/w), cholesterol (4.95 mg, 9.9% w/w), DOPE (7.0 mg, 14% w/w), DC-Chol (14.0 mg, 28% w/w), and DSPE-PEG 2k (0.05 mg, 0.1% w/w), has particle size of about 100˜130 nm, and is positively charged is prepared. To evaluate the properties of a HA-DAB-siRNA/CSLN complex, HA-DAB-siRNA (amount corresponding to 1 nmol based on siRNA) and various w/w ratios of CSNL were mixed to form complexes, and electrophoresis was conducted at 1% Agarose gel and the results are shown in FIG. 11.

FIG. 11 shows the results of confirming the formation of HA-DAB-siRNA/CSLN complexes through agarose gel electrophoresis.

As shown in FIG. 11, a HA-DAB-siRNA/CSLN complex was formed at the w/w ratio of CSLN/siRNA of 20 or more.

The HA-DAB-siRNA/CSLN complex was diluted in PBS 1 ml, the particle size and the surface charge were measured using a DLS analyzer (Zetasizer Nano, Malvern Instrument Co., UK) and the results are shown in Table 1 and Table 2, respectively.

TABLE 1

| Sample Name | Z-Ave size(nm) | PDI | ZP (mV) |
|---|---|---|---|
| siRNA/CSLN w/w 20_1 | 300.2 | 0.290 | +40.9 |
| siRNA/CSLN w/w 20_2 | 283.1 | 0.331 | +42.0 |
| siRNA/CSLN w/w 20_3 | 297.0 | 0.320 | +46.7 |
| HA-siRNA/CSLN w/w 20_ 1 | 350.4 | 0.239 | −19.2 |
| HA-siRNA/CSLN w/w 20_ 2 | 297.2 | 0.251 | −17.1 |
| HA-siRNA/CSLN w/w 20_ 3 | 418.7 | 0.268 | −16.8 |

TABLE 2

| Sample Name | Z-Ave (d · nm) | PDI | ZP (mV) |
|---|---|---|---|
| siRNA/CSLN w/w 30_1 | 286.6 | 0.219 | +46.2 |
| siRNA/CSLN w/w 30_2 | 302.3 | 0.312 | +47.3 |
| siRNA/CSLN w/w 30_3 | 268.8 | 0.170 | +46.8 |
| HA- siRNA/CSLN w/w 30_1 | 326.3 | 0.38 | −13.6 |
| HA- siRNA/CSLN w/w 30_2 | 322.5 | 0.321 | −12.8 |
| HA- siRNA/CSLN w/w 30_3 | 361.1 | 0.271 | −13.0 |

Table 1 and Table 2 respectively show the results of measuring the particle size and the surface charge of the HA-DAB-siRNA/CSLN complexes at w/w ratios of 20 and 30. As the result, at w/w ratios of 20 and 30, the complexes respectively have particle size of 293.4 nm and 285.9 nm, and surface charge of average +43.2 mV and +46.76 mV. Although the HA-siRNA/CSLN complex has slightly larger average size than the siRNA/CSLN complex, the size of firm core is considered to be important, and since the HA-siRNA/CSLN complex is weakly negatively charged, it is anticipated to minimize non-specific bonding to blood protein, and the like when delivered in the body.

### Experimental Example 7

### Evaluation of Cytotoxicity and Gene Inhibition Efficiency Of HA-DAB-siRNA/Cationic Solid Lipid Nanoparticle Complex

To evaluate the gene inhibition efficiency of a HA-DAB-siRNA/CSLN complex, cells were treated with HA-DAB-siRNA/CSLN and the degree of gene expression was measured. Specifically, Hela cells that express mouse factor VII (mFVII) and are cultured in RPMI medium (GIBCO-BRL, NY, USA) containing 10% fetal bovine serum and 100 U/mL penicillin-streptomycin were seeded into a 96-well plate at 5×10³ cells/well, and cultured at 37° C. and 5% CO₂ for 12 hours.

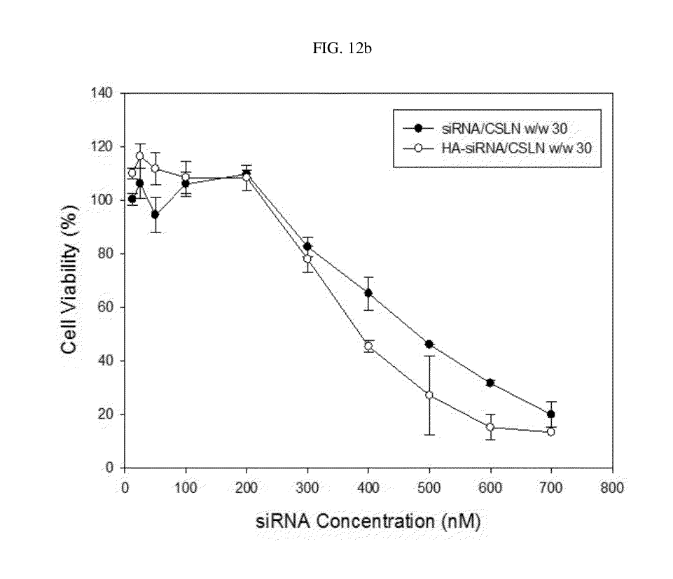

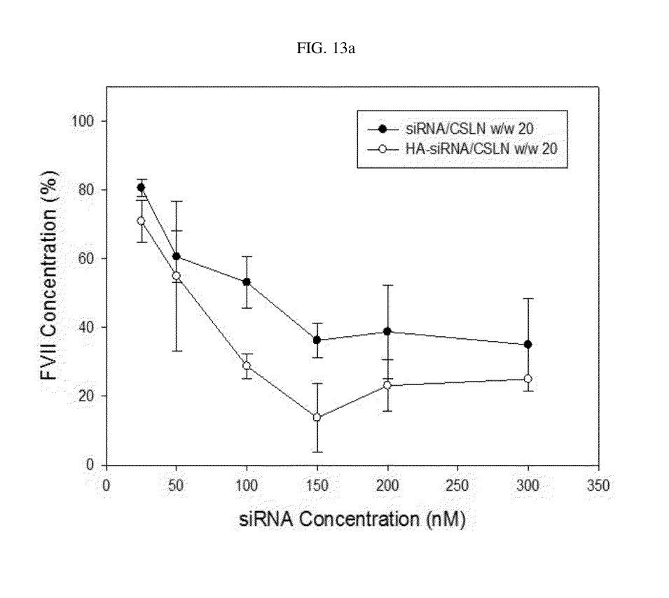

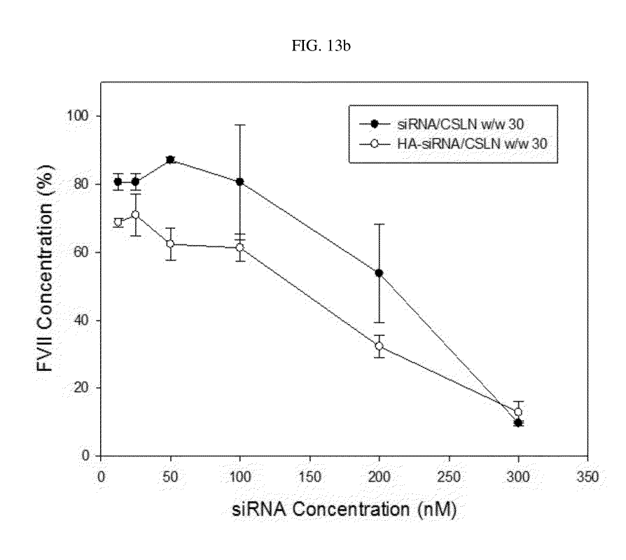

To form a complex, the HA-DAB-siRNA complex prepared in Example 1-2 and CSLN were mixed in PBS at w/w ratios of 20 and 30 for 15 minutes, and stored. And then, the above prepared cells were treated therewith at various concentrations and additionally cultured for 24 hours. And then, supernatant was collected, and the concentration of mFVII was analyzed using BIOPHEN VII Ref A221304 (Aniara, Ohio, USA). New RPMI medium was put in remaining cells, the degree of cytotoxicity was analyzed by MTT assay, and the results of cytotoxicity are shown in FIGS. 12a and 12b, and the results of gene inhibition efficiency are shown in FIGS. 13a and 13b. As control, siRNA/CSLN complexes with w/w ratios of 20 and 30 were compared.

FIGS. 12a and 12b show the results of confirming the toxicity of the HA-DAB-siRNA/CSLN complex by MTT using Hela cell line, and FIGS. 13a and 13b show the results of measuring the concentration of mFVII secreted in the supernatant after treated with the HA-DAB-siRNA/CSLN complex.

As shown in FIGS. 12a, 12b, 13a and 13b, the HA-siRNA/CSLN complex with w/w ratio of 20 has decreased toxicity and increased efficiency compared to the complex with w/w ratio of 30. The HA-siRNA/CSLN complex with w/w ratio of 20 has 1.38 times higher LC 50 (Lethal Concentration 50)/IC 50 (Inhibitory concentration 50) compared to the siRNA/CSLN complex with w/w ratio of 20.

What is claimed is:

1. A hyaluronic acid-alkylenediamine conjugate where the hyaluronic acid is connected with C1-10 alkylenediamine by a peptide bond.

2. The hyaluronic acid-alkylenediamine conjugate according to claim 1, wherein the peptide bond is formed between the amine group of the alkylenediamine and the carboxylic acid group of the hyaluronic acid.

3. The hyaluronic acid-alkylenediamine conjugate according to claim 1, wherein the alkylenediamine is C4-8 alkylenediamine, and the hyaluronic acid has an average molecular weight of 10,000 to 3,000,000.

4. The hyaluronic acid-alkylenediamine conjugate according to claim 1, which is represented by the following Chemical Formula 3: 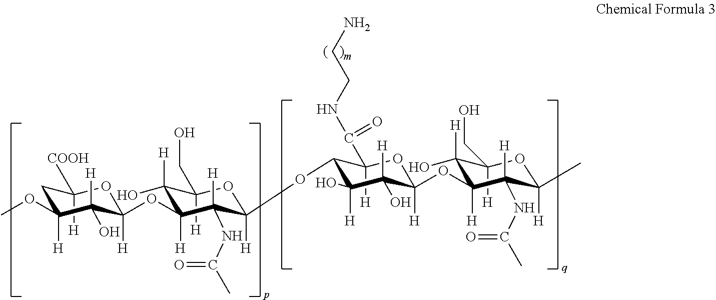
wherein, m is an integer of 1 to 10, and p and q are independently an integer of 16 to 2500.

5. A complex comprising a hyaluronic acid-alkylenediamine conjugate and a nucleic acid where the C1-10 alkylenediamine connected with the hyaluronic acid by a peptide bond, is bonded directly or through a linker to the nucleic acid containing a thiol group at an amine group of the alkylenediamine.

6. The hyaluronic acid-alkylenediamine-nucleic acid complex according to claim 5, wherein the linker comprises a first functional group that can be bonded to the amine group of the alkylenediamine, and a second functional group that can be bonded to the thiol group of the nucleic acid.

7. The hyaluronic acid-alkylenediamine-nucleic acid complex according to claim 6, wherein the first functional group of the linker is a carboxylic acid group, and the second functional group is a thiol group.

8. The hyaluronic acid-alkylenediamine-nucleic acid complex according to claim 6, wherein the linker compound is succinimidyl 3-(2-pyridyldithio)propionate (SPDP).

9. The hyaluronic acid-alkylenediamine-nucleic acid complex according to claim 5, wherein the thiol group is introduced at the 3′ end of the nucleic acid.

10. The hyaluronic acid-alkylenediamine-nucleic acid complex according to claim 5, wherein the alkylenediamine is C4-8 alkylenediamine, and the hyaluronic acid has average molecular weight of 10,000 to 3,000,000.

11. The hyaluronic acid-alkylenediamine-nucleic acid complex according to claim 5, wherein the hyaluronic acid-alkylenediamine complex has a structure of the following Chemical Formula 4: 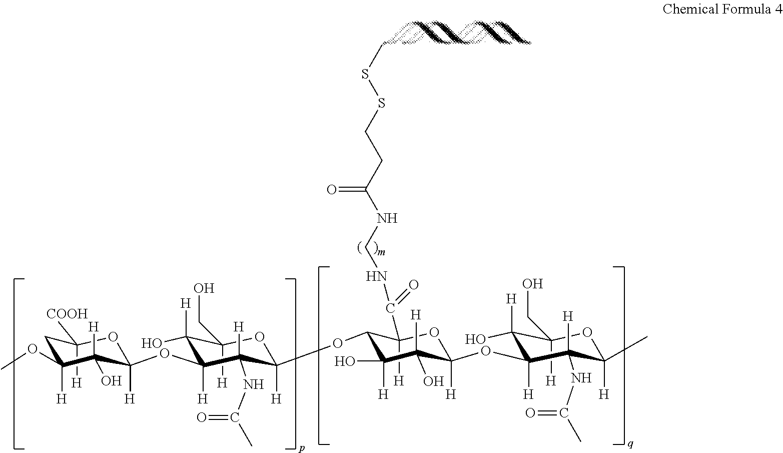
wherein, m is an integer of 1 to 10, and p and q are independently an integer of 16 to 2500.

12. The hyaluronic acid-alkylenediamine-nucleic acid complex according to claim 5, further comprising a cationic material.

13. The hyaluronic acid-alkylenediamine-nucleic acid complex according to claim 12, wherein the cationic material is at least one selected from the group consisting of polyethyleneimine, poly(L-lysine), polymethacrylate, chitosan, poly cationic dendrimers, cationic peptide, quantum dot, gold nanoparticles, silica nanoparticles, carbon derivative nanoparticles, and solid lipid nanoparticles.

14. The hyaluronic acid-alkylenediamine-nucleic acid complex according to claim 12, wherein the cationic material is low density lipoprotein-like (LDL-like) nanoparticle of a core-shell structure comprising a core comprising cholesteryl ester and triglyceride; and a shell comprising cholesterol, fusogenic lipid, cationic lipid, and a lipid-PEG (polyethyleneglycol) conjugate.

15. A method for in vivo delivery of nucleic acid, comprising administering the hyaluronic acid-alkylenediamine-nucleic acid complex according to claim 5 to a subject in need.

16. The method for in vivo delivery of nucleic acid according to claim 15, wherein the thiol group is introduced at the 3′ end of the nucleic acid.

17. The method for in vivo delivery of nucleic acid according to claim 15, wherein the alkylenediamine is C4-8 alkylenediamine, and the hyaluronic acid has average molecular weight of 10,000 to 3,000,000.

18. The method for in vivo delivery of nucleic acid according to claim 15, wherein hyaluronic acid-alkylenediamine-nucleic acid complex further comprise cationic material.

19. The method for in vivo delivery of nucleic acid according to claim 18, wherein the cationic material is at least one selected from the group consisting of polyethyleneimine, poly(L-lysine), polymethacrylate, chitosan, poly cationic dendrimers, cationic peptide, quantum dot, gold nanoparticles, silica nanoparticles, carbon derivative nanoparticles, and solid lipid nanoparticles.

20. The method for in vivo delivery of nucleic acid according to claim 18, wherein the cationic material is low density lipoprotein-like (LDL-like) nanoparticle of a core-shell structure comprising a core comprising cholesteryl ester and triglyceride; and a shell comprising cholesterol, fusogenic lipid, cationic lipid, and a lipid-PEG (polyethyleneglycol) conjugate.
